(19) United States
(12) Patent Application Publication
(10) Pub. No.: US 2018/0364238 A1
(43) Pub. Date: Dec. 20, 2018

(54) Method for Detecting Cancer Stem Cells

(71) Applicant: UNIVERSITE DE LIMOGES, Limoges Cedex (FR)

(72) Inventors: Aurélie Lacroix, Panazol (FR); Muriel Mathonnet, Panazol (FR); Fabrice Lalloue, Isle (FR); Marie-Odile Jauberteau, Limoges (FR)

(21) Appl. No.: 15/780,675

(22) PCT Filed: Dec. 2, 2016
(86) PCT No.: PCT/FR2016/053196

§ 371 Date: Jun. 1, 2018

(30) Foreign Application Priority Data
Dec. 2, 2015 (FR) 1561763

(51) Int. Cl. G01N 33/574

(52) U.S. Cl. CPC G01N 33/57419; G01N 33/57492; G01N 2800/56; G01N 2800/52; G01N 2333/4724

(57) ABSTRACT

The invention relates to the use of a lectin that recognizes the fucose α 1-2 galactose unit, as a means for labeling colorectal cancer stem cells, for the detection and/or quantification of said colorectal cancer stem cells, and the use of said lectin in a method for diagnosing the aggressiveness, recurrence risk and a prognostic value in order to adapt colorectal cancer treatment.

This invention relates to the field of detecting cancer stem cells, particularly colorectal cancer stem cells.

Colorectal cancer (CRC) is the third most frequent disease in the world. Like any cancer, it can be summed up as an abnormal cell proliferation in a healthy tissue, in this case the colonic mucosa, causing the appearance of a tumor mass. One of the theories put forward to explain tumor development as well as the resistance mechanisms and recurrences lies in the existence of cancer stem cells. The therapeutic escape of the tumor from radio- and chemo-therapy treatments depends on the presence of these cells within the tumor. Consequently, the detection of these cells in the tumor tissue constitutes a means of defining the level of aggressiveness of the tumor. The characterization of specific biomarkers of cancer stem cells is therefore of huge diagnostic and prognostic interest in the treatment of cancer. However, no specific markers of cancer stem cells (CSCs) currently exist that allow them to be distinguished with certainty from other tumor cells.

Due to their small number and the absence of specific markers, the major difficulties in the study of CSCs lie in their isolation and characterization.

A need therefore exists for a method that allows colorectal cancer stem cells to be detected in a colorectal tumor.

The present invention meets that need. The inventors of the present invention have identified, as a marker of colorectal cancer stem cells, specific glycans expressed on the surface of this population of cells. The recognition of these specific markers by appropriate means makes it possible to detect and quantify the colorectal cancer stem cells within a colorectal tissue in which non-cancer non-stem cancer cells, cancer non-stem cells and non-cancer stem cells are present.

In a first aspect, the present invention concerns the use of a lectin for detecting and/or quantifying colorectal cancer stem cells (CSC) in vitro and potentially a second means of labeling colorectal cancer stem cells.

In a second aspect, the present invention concerns a method for detecting and/or quantifying colorectal cancer stem cells in a colorectal biological sample.

In a third aspect, the present invention relates to a method for diagnosing the recurrence risk and/or aggressiveness of a colorectal cancer in order to define a prognostic value so as to adapt colorectal cancer treatment.

In a fourth aspect, the present invention concerns a kit comprising a lectin and a second means of labeling colorectal cancer stem cells, as well as the use of said kit for detecting and/or quantifying colorectal cancer stem cells, specifically for diagnosing the recurrence risk and/or the aggressiveness of a colorectal cancer so as to define a prognostic value in order to adapt the treatment of a colorectal cancer.

A first subject-matter of the present invention thus relates to the use of a means that allows the fucose α 1-2 galactose unit to be specifically recognized, in particular a lectin that recognizes the fucose α 1-2 galactose unit, for detecting and/or quantifying colorectal cancer stem cells.

The present invention relates in particular to the use of a means that allows the fucose α 1-2 galactose unit to be specifically recognized, particularly a lectin that recognizes the fucose α 1-2 galactose unit, for detecting and/or quantifying in vitro colorectal cancer stem cells in a colorectal biological sample.

Colorectal cancer stem cells are a population of quiescent cells, capable of self-renewal and resistant to numerous substances used in chemotherapy. They are also known as cancer tumor-initiating cells (TIC). Colorectal cancer stem cells have already been described and have been the subject of reviews, for example in Vaiopoulos et al. S, Stem Cells 2012 (30), 363-371 and Ricci-Vitiani et al. J. Mol. Med. 2009, 87 (11), 1097-1104.

The present invention also relates the use, as a first labeling means, of a lectin that recognizes the fucose α 1-2 galactose unit in order to implement a method for detecting and/or quantifying colorectal cancer stem cells in vitro in a colorectal biological sample.

Within the meaning of the present invention, a “means of labeling colorectal cancer stem cells” means a substance capable of binding specifically to a marker expressed on the surface of colorectal cancer stem cells. The labeling means can in particular be an antibody directed against an antigenic determinant, such as a glycoprotein, a protein or a glycan.

The lectin that recognizes the fucose α 1-2 galactose unit according to the present invention is advantageously chosen from Ulex Europaeus Agglutinin I (UEA-I) and Trichosanthes Japonica Agglutinin II (TJA-II). Preferably, from Ulex Europaeus Agglutinin I.

In order to guarantee that the cells labeled by the lectin that recognizes the fucose α 1-2 galactose unit are colorectal cancer stem cells, it is advantageous to use, in addition to the lectin that recognizes the fucose α 1-2 galactose unit, a second means of labeling colorectal cancer stem cells. This second means of labeling can be specific to cancer- or non-cancer stem cells.

Examples of colorectal cancer stem cell markers include CD133, CD44, CD166 (ALCAM), CD24, CD26, CD29, EpCAM, Oct-4 and Sox-2.

Preferably, the second means of labeling colorectal cancer stem cells is an anti-OCT4 antibody (octamer-binding transcription factor 4, coded by the gene POU5F1).

The present invention thus also relates to the use of a lectin that recognizes the fucose α 1-2 galactose unit, advantageously chosen from Ulex Europaeus Agglutinin I (UEA-I) and Trichosanthes Japonica Agglutinin II (TJA-II) for detecting and/or quantifying colorectal cancer stem cells.

According to an embodiment, the colorectal cancer stem cells can be labeled by a lectin that recognizes the fucose α 1-2 galactose unit, an anti-OCT4 antibody and at least one lectin that recognizes the T antigen.

The lectin that recognizes the T antigen can in particular be chosen from Agaricus Bisporus Agglutinin (ABA), Amaranthus Caudatus Lectin (ACA) and Jacalin.

To label colorectal cancer stem cells, a mixture of lectins that recognize the T antigen can also be used, specifically a mixture of two lectins chosen from ABA and ACA; ABA and Jacalin; ACA and Jacalin. Advantageously, the lectins are UEA-1, Jacalin and ACA.

Advantageously, the mixture of three lectins consists of a lectin that recognizes the fucose α 1-2 galactose unit:Jacalin:ABA or ACA, in a molar ratio of 625 to 12500:16 to 320:1 to 20, advantageously from 5000 to 7000:50 to 250:5 to 15, specifically 6250:160:10.

Yet more advantageously, the lectin that recognizes the fucose α 1-2 galactose unit is UEA-1 and the lectins that recognize the T antigen are Jacalin and ACA in a molar ratio of UEA-1:Jacalin:ACA of 6250:160:10.

In order to detect and quantify the colorectal cancer stem cells, the means for labeling the colorectal cancer stem cells must be capable of being seen in the biological sample.

The labeling means are therefore directly or indirectly bound to a revelation means.

Within the meaning of the present invention, “revelation means” signifies a substance or a collection of substances capable of recognizing the labeling means and transmitting a signal that can be detected in the tissue, for example by direct visualization or spectrophotometry.

“Directly bound” within the meaning of the present invention means that the revelation means is covalently bound to the means of labeling the colorectal cancer cells. The revelation means can, for example be a chromophore, a fluorophore or an enzyme capable of reducing a chromogenic substrate, bound covalently to the labeling means.

“Indirectly bound” within the meaning of the present invention means that the revelation means is covalently bound to a secondary substance, said secondary substance being capable of specifically recognizing the labeling means. The revelation means can, for example, be covalently bound to avidin, streptavidin or an anti-biotin antibody and the labeling means can be biotinylated.

The present invention thus also relates to the use of a lectin that recognizes the fucose α 1-2 galactose unit bound to a revelation means A in order to implement a method for detecting and/or quantifying the colorectal cancer stem cells in a colorectal biological sample.

The revelation means A can be a chromophore, a fluorophore, an antibody that recognizes the fucose α 1-2 galactose unit bound directly or indirectly to a fluorophore or a chromophore, or an enzyme capable of reducing a chromogenic substrate B.

Advantageously, the revelation means A is an enzyme capable of reducing a chromogenic substrate B.

In one embodiment, the lectin that recognizes the fucose α 1-2 galactose unit is biotinylated and the revelation means A is bound to streptavidin. Advantageously, the revelation means A is an enzyme capable of reducing a chromogenic substrate, specifically a horseradish peroxidase. Said enzyme capable or reducing a chromogenic substrate B can be incorporated into a signal amplification system. Such signal amplification systems are available on the market, for example from the Leica or ThermoFisher Scientific companies.

The chromogenic substrate B depends on the class of enzyme used. In the case of horseradish peroxidase, it can for example be a chromogenic substrate chosen from the group consisting of 3,3′,5,5′-tetramethylbenzidine (TMB), ortho-phenylenediamine (OPD), 3,3′-diaminobenzidine (DAB), 10-acetyl-10H-Phenoxazine-3,7-diol (AmplexRed®), homovanillic acid, luminol, 3-amino-9-ethylcarbazole (AEC) and 2,2′-azino-bis(3-ethylbenzothiazoline-6-sulfonic acid (ABTS).

Advantageously, the chromogenic substrate B is 3,3′-diaminobenzidine (DAB).

The second labeling means, preferably an anti-OCT4 antibody, is bound to a revelation means C, advantageously chosen from a chromophore, a fluorophore or an enzyme capable of reducing a chromogenic substrate D.

Said revelation means C can be bound directly or indirectly to the labeling means of the colorectal cancer stem cells.

Advantageously, the revelation means C is bound to a secondary antibody that recognizes the second labeling means. For example, the labeling means can be an antibody produced in a first animal species and the antibody to which the revelation means C is bound in an antibody produced in another animal species that recognizes an epitope specific to the animal species of the antibody used as a labeling means, specific to a sequence of immunoglobulin chains. Alternatively, the revelation means C can be bound to a tertiary antibody that recognizes a secondary antibody that recognizes the second labeling means, specifically an anti-OCT4 antibody.

In one embodiment, the second colorectal cancer stem cell labeling means is an anti-OCT4 antibody and the revelation means C is bound to an antibody that recognizes the anti-OCT4 antibody. Advantageously, said revelation means C is an enzyme capable of reducing a chromogenic substrate D, specifically an alkaline phosphatase. Said enzyme capable of reducing a chromogenic substrate D can specifically be incorporated into a signal amplification system. Such signal amplification systems are available on the market, for example from the Leica or Thermofisher Scientific companies. In a specific embodiment, the anti-OCT4 antibody is an antibody produced in rabbits and the revelation means is bound to a rabbit anti-immunoglobulin antibody, for example of a mouse.

The chromogenic substrate D depends on the class of enzyme used. In the case of an alkaline phosphatase, it may for example be a chromogenic substrate chosen from the group consisting of 5-bromo, 4-chloro, 3-indolylphosphate (BCIP), Nitro-Blue tetrazolium (NBT), a BCIP/NBT mixture, 4-Nitrophenyl phosphate (p-NPP), 3-indoxyl phosphate, 7-bromo-N-(2-methoxyphenyl)-3-(phosphonooxy)-2-naphthalenecarboxamide and 3-amino-4-methoxybenzamide (Fast RED®).

Advantageously, the chromogenic substrate is 3-amino-4-methoxybenzamide (Fast RED®) or a BCIP/NBT mixture.

Advantageously, the chromogenic substrate B and the chromogenic substrate D result in the formation of chromophores of different colors, allowing the cells labeled by the first labeling means and the cells labeled by the second labeling means to be differentiated.

The present invention thus also relates to the use of a biotinylated lectin that recognizes the fucose α 1-2 galactose unit and of an anti-OCT4 antibody, streptavidin horseradish peroxidase conjugate and an antibody that recognizes the anti-OCT4 antibody bound to an alkaline phosphatase in order to implement a method for detecting and/or quantifying colorectal cancer stem cells. Advantageously, the chromogenic substrate B is DAB and the chromogenic substrate D is Fast RED® or BCIP/NBT.

The use as a revelation means A of an enzyme capable of reducing a chromogenic substrate B and as a revelation means C of an enzyme capable of reducing a chromogenic substrate D is particularly advantageous in an immunohistochemical method, particularly for use on histological sections.

In a specific embodiment, the present invention relates to the use of a lectin that recognizes the fucose α 1-2 galactose unit as defined above in an immunohistochemical detection method.

In this particular embodiment, the biological sample is a histological section of a colorectal biological sample, specifically of a tumor resection or a colorectal biopsy.

In this particular embodiment, the revelation means A can be bound covalently to the lectin that recognizes the fucose α 1-2 galactose unit. The revelation means A can also be bound to avidin, streptavidin or an anti-biotin antibody and the lectin that recognizes the fucose α 1-2 galactose unit is biotinylated.

In this particular embodiment, the revelation means C can be covalently bound to the second labeling means. The revelation means C can also be covalently bound to a tertiary antibody that recognizes a secondary antibody that recognizes the second labeling means. Preferably, the second labeling means is an anti-OCT4 antibody and the revelation means C is covalently bound to the anti-OCT4 antibody or covalently bound to a secondary antibody that recognizes the anti-OCT4 antibody. The revelation means C can also be covalently bound to a tertiary antibody that recognizes a secondary antibody that recognizes the anti-OCT4 antibody.

In this particular embodiment, the present invention preferably relates to the use of a biotinylated lectin that recognizes the fucose α 1-2 galactose unit, an enzyme capable of reducing a chromogenic substrate B bound to streptavidin, an anti-OCT4 antibody, an enzyme capable of reducing a chromogenic substrate D bound to a secondary antibody that recognizes the anti-OCT4 antibody, for detecting and/or quantifying colorectal cancer stem cells in a histological section of a colorectal tissue.

Most preferably of all, the biotinylated lectin that recognizes the fucose α 1-2 galactose unit is UEA-1, the second labeling means is an anti-OCT4 antibody, the revelation means A is a horseradish peroxidase, the chromogenic substrate B is diaminobenzidine, the revelation means C is an alkaline phosphatase bound to a secondary antibody and the chromogenic substrate D is 3-amino-4-methoxybenzamide (Fast RED®).

A second subject-matter of the present invention relates to a method for detecting and/or quantifying colorectal cancer stem cells in vitro in a colorectal biological sample, comprising a revelation step of a lectin that recognizes the fucose α 1-2 galactose unit in a colorectal biological sample labeled by said lectin.

“Revelation step” within the meaning of the present invention means the step aimed at visually detecting, by spectroscopic or spectrophotometric methods, the lectin that recognizes the fucose α 1-2 galactose unit bound to the colorectal cancer stem cells, after placing said lectin in contact with the biological sample.

More particularly, the present invention relates to a method for detecting and/or quantifying in vitro as defined above, comprising the steps of:
(a) labeling the colorectal cancer stem cells with a lectin that recognizes the fucose α 1-2 galactose unit in said biological sample, in order to obtain labeled colorectal cancer stem cells in said biological sample,
(b) revealing said lectin in the colorectal biological sample,
(c) detecting and/or quantifying the colorectal cancer stem cells labeled by said lectin in said biological tissue.

The labeling step is preceded by conventional steps intended to prepare the biological sample, such as washing the tissue, using a buffer designed to unmask the antigens (in the case of double labeling, the pH is advantageously 6).

The labeling step is advantageously performed in a diluent having a saturation power and capable of preventing non-specific bindings (for example a PBS-BSA diluent).

The labeling step is implemented at a temperature of between 10 and 30° C., advantageously for an incubation time ranging between 10 and 30 minutes.

The present invention thus advantageously relates to a method for detecting and/or quantifying colorectal cancer stem cells in a histological section of colorectal tissue, comprising the steps of:
(a2) labeling the colorectal cancer stem cells of the histological section with a biotinylated lectin that recognizes the fucose α 1-2 galactose unit, in order to obtain labeled colorectal cancer stem cells in said histological section,
(b2) placing the histological section obtained at step (a2) in contact with the revelation means A bound to streptavidin, avidin or an anti-biotin antibody,
(c2) revealing the lectin that recognizes the fucose α 1-2 galactose unit,
(d2) detecting and/or quantifying the colorectal cancer stem cells labeled by said lectin in histological section.

Advantageously, the method described above can be implemented at step (a) with a lectin that recognizes the fucose α 1-2 galactose unit and at least one lectin that recognizes the T antigen, advantageously chosen from ACA, ABA and Jacalin, more advantageously with a mixture of lectins that recognize the T antigen chosen from a mixture of ABA and ACA, a mixture of ABA and Jacalin and a mixture of ACA and Jacalin, advantageously, ACA and Jacalin.

Advantageously, the mixture of lectins consists of a lectin that recognizes the fucose α 1-2 galactose unit:Jacalin:ABA or ACA, in a molar ratio of 625 to 12500:16 to 320:1 to 20, advantageously 5000 to 7000:50 to 250:5 to 15, specifically 6250:160:10.

Particularly advantageously, step (a) is implemented with a mixture of UEA-1, ACA and Jacalin, preferably in a UEA-1:Jacalin:ACA ratio of 6250:160:10.

The method described above can be preceded by a step of labeling with a second means of labeling the cancer stem cells as defined above. In particular, the method described above can be preceded by a step of labeling by an anti-OCT4 antibody.

The present invention thus concerns a method comprising, prior to step (a) or (a2) of labeling by the lectin that recognizes the fucose α 1-2 galactose unit, the steps of:
(a′1) labeling the colorectal cancer stem cells of said colorectal biological sample with a second labeling means, preferably of an anti-OCT4 antibody, in order to obtain colorectal cancer stem cells labeled by an anti-OCT4 antibody in said biological sample,
(a′2) revealing the second labeling means, preferably the anti-OCT4 antibody.

The labeling step is preceded by conventional steps designed to prepare the biological sample, such as washing the tissue, using a buffer designed to unmask the antigens (in the case of double labeling, the pH is advantageously 6).

The labeling step is advantageously performed in a diluent having a saturation power and capable of preventing non-specific adhesions (for example a PBS-BSA diluent).

The labeling step is implemented at a temperature of between 10 and 30° C., advantageously for an incubation time ranging from 10 to 30 minutes.

The present invention thus also relates to a method as described above, comprising the steps of:
(a3) labeling the colorectal cancer stem cells with a second labeling means of colorectal cancer stem cells, preferably an anti-OCT4 antibody, in a histological section of a colorectal tissue, in order to obtain colorectal cancer stem cells labeled by said second labeling means in said histological sample,
(b3) placing the histological section obtained at step (a3) in contact with a secondary antibody that recognizes the second labeling means, preferably the anti-OCT4 antibody, bound to a revelation means C,
(c3) revealing the second labeling means,
(d3) labeling the colorectal cancer stem cells of the histological section obtained at step (c3) with a biotinylated lectin that recognizes the fucose α 1-2 galactose unit, and the possible at least one biotinylated lectin that recognizes the T antigen, in order to obtain cancer stem cells labeled by lectin that recognizes the fucose α 1-2 galactose unit and possibly labeled by the at least one lectin that recognizes the T antigen in said histological section,
(e3) placing the histological section obtained at step (d3) in contact with a revelation means A bound to streptavidin, avidin or an anti-biotin antibody,
(f3) revealing the lectin that recognizes the fucose α 1-2 galactose unit and the possible at least one lectin that recognizes the T antigen,
(g3) detecting and/or quantifying the colorectal cancer stem cells labeled by the lectin that recognizes the fucose α 1-2 galactose unit and the second labeling means, preferably the anti-OCT4 antibody in the histological section.

In a particular embodiment, the present invention relates to a method for detecting and/or quantifying colorectal cancer stem cells in a colorectal histological section comprising the steps of:
(a4) labeling colorectal cancer stem cells with an anti-OCT4 antibody in a histological section of a colorectal tissue, in order to obtain colorectal stem cells labeled by an anti-OCT4 antibody in said histological section,
(b4) placing the histological section obtained in step (a4) in contact with a secondary antibody that recognizes the anti-OCT4 antibody bound to an enzyme capable of reducing a chromogenic substrate,
(c4) revealing the anti-OCT4 antibody with a chromogenic substrate,
(d4) labeling the colorectal cancer stem cells of the histological section obtained at step (c4) with a biotinylated lectin that recognizes the T antigen, in order to obtain colorectal cancer stem cells labeled by lectin that recognizes the fucose α 1-2 galactose unit and the possible at least one lectin that recognizes the T antigen in said histological section,
(e4) placing the histological section obtained at step (d4) in contact with streptavidin, avidin or an anti-biotin antibody bound to an enzyme capable of reducing a chromogenic substrate,
(f4) revealing lectin that recognizes the fucose α 1-2 galactose unit and the possible at least one lectin that recognizes the T antigen with a chromogenic substrate,
(g4) detecting and/or quantifying colorectal cancer stem cells labeled by lectin that recognizes the fucose α 1-2 galactose unit, possibly the at least one biotinylated lectin that recognizes the T antigen, and the anti-OCT4 antibody in the histological section.

In a preferred embodiment, the present invention relates to a method for detecting and/or quantifying colorectal cancer stem cells in a colorectal histological section, comprising the steps of:
(a5) marking the colorectal cancer stem cells with an anti-OCT4 antibody in a histological section of a colorectal tissue, in order to obtain colorectal stem cells labeled by an anti-OCT4 antibody in said histological section,
(b5) placing the histological section obtained in step (a5) in contact with a secondary antibody that recognizes the anti-OCT4 antibody bound to an alkaline phosphatase,
(c5) revealing the anti-OCT4 antibody with FastRED® or a BCIP/NBT mixture,
(d5) labeling the colorectal cancer stem cells of the histological section obtained at step (c5) with biotinylated UEA-1, and possibly biotinylated ACA and biotinylated Jacalin in a UEA-1:Jacalin:ACA ratio of 6250:160:10, to obtain colorectal cancer stem cells labeled by UEA-1 and possibly ACA and Jacalin,
(e5) placing the histological section obtained at step (d5) in contact with the streptavidin bound to the horseradish peroxidase,
(f5) revealing UEA-1 and possibly ACA and Jacalin with DAB,
(g5) detecting and/or quantifying colorectal cancer stem cells labeled by UEA-1 and the anti-OCT4 antibody and possibly ACA and Jacalin in the histological section.

In another embodiment, the general methodology described in the present application can be implemented by using flow cytometry to detect and/or quantify colorectal cancer stem cells. In one embodiment, cancer stem cells can be detected and/or quantified by flow cytometry. This method also enables the colorectal cancer stem cells to be isolated by using the appropriate equipment.

The biological sample can be a biopsy of a colorectal tumor tissue in which the cells have been dissociated from one another beforehand, for example by means of the product Liberase™, sold by the Roche Diagnostic company, which enables the dissociation of the tissue without affecting the surface glycans.

In order to detect and/or quantify the cells by flow cytometry, the lectin that recognizes the fucose α 1-2 galactose unit is directly or indirectly bound to a fluorophore E. The second labeling means, preferably an anti-OCT4 antibody, is directly or indirectly bound to a fluorophore F, for example to a secondary antibody that recognizes the anti-OCT4 antibody. Advantageously, the fluorophores E and F differ from one another. Preferably, the fluorophore E is Alexa 488 bound to streptavidin and the lectin that recognizes the fucose α 1-2 galactose unit is biotinylated and the fluorophore F is Alexa 633 directly bound to the anti-OCT4 antibody.

The present invention also relates to a method for detecting and/or quantifying colorectal cancer stem cells as described above, in a colorectal biological sample, in which the detection step is implemented by flow cytometry, with fluorophores as the revelation means.

As the method according to the present enables colorectal cancer stem cells to be detected and quantified, it is particularly suitable for the diagnosis of colorectal cancer, particularly by means of an anatomopathological or histopathological examination.

Cancer stem cells differ from other cells that comprise tumors. They are capable of self-renewing and are resistant to conventional chemotherapy and radiotherapy treatments. Cancer stem cells would therefore cause tumor recurrence after a conventional chemotherapy/radiotherapy that is nonetheless capable of eliminating tumors. The detection of these cells in a tumor tissue is therefore of fundamental interest in assessing the recurrence risk in a patient treated by chemotherapy or radiotherapy. Quantification of cancer stem cells is also crucial for determining the aggressiveness of a cancer. Furthermore, the detection of cancer stem cells in a tumor tissue allows a prognosis to be established during treatment, thus offering the possibility of changing the treatment in order to target these cells more effectively and thus reduce the aggressiveness of the tumor and the recurrence risks.

The present invention thus relates to the use of a lectin that recognizes the fucose α 1-2 galactose unit, and possibly an anti-OCT4 antibody, for the in vitro diagnosis of the recurrence risk of a colorectal cancer and/or the aggressiveness of a colorectal cancer to define a prognostic value in order to adapt the treatment of a colorectal cancer.

In one embodiment, labeling is achieved using a lectin that recognizes the fucose α 1-2 galactose unit, advantageously UEA-1, an anti-OCT4 antibody and at least one lectin that recognizes the T antigen, advantageously chosen from ACA, ABA and Jacalin or a mixture of lectins that recognize the T antigen chosen from an AVA and ACA mixture, and an ABA and Jacalin mixture and an ACA and Jacalin mixture, advantageously ACA and Jacalin. Advantageously, the UEA-1:Jacalin:ACA ratio is 6250:160:10.

The present invention relates more particularly to an in vitro diagnostic method for the recurrence risk and/or aggressiveness and/or prognostic value of a colorectal cancer treatment comprising the steps of:
(a) labeling the colorectal cancer stem cells in a colorectal biological sample with a lectin that recognizes the fucose α 1-2 galactose unit, in order to obtain colorectal cancer stem cells labeled by lectin that recognizes the fucose α 1-2 galactose unit in said biological sample,
(b) revealing colorectal cancer stem cells in the colorectal tissue,
(c) detecting and/or quantifying cancer stem cells labeled by the lectin that recognizes the fucose α 1-2 galactose unit,
(d) deducing the recurrence risk and/or aggressiveness and/or prognostic value of the colorectal cancer treatment on the basis of the quantity of colorectal cancer stem cells.

Advantageously, the present invention is implemented by using a lectin that recognizes the fucose α 1-2 galactose unit and a second means of labeling colorectal cancer cells, preferably an anti-OCT4 antibody.

The inventors have in fact shown that a double labeling has resulted in optimum sensitivity and specificity, thus drastically reducing the risk of false positives that could result from labeling cancer non-stem cells or non-cancer stem cells by the lectin that recognizes the fucose α 1-2 galactose unit.

The present invention thus also relates to an in vitro diagnostic method of the recurrence risk and/or aggressiveness in order to define a prognostic value for therapeutic adaptation of a colorectal cancer comprising the steps of:
(a6) labeling colorectal cancer stem cells with a second labeling means, preferably an anti-OCT4 antibody in a histological section of a colorectal tissue, in order to obtain labeled colorectal cancer cells in said histological section,
(b6) placing the histological section obtained in step (a6) in contact with a secondary antibody that recognizes the second labeling means bound to a revelation means C,
(c6) revealing the second labeling means, preferably the anti-OCT4 antibody,
(d6) labeling the colorectal cancer stem cells of the histological section obtained at step (c6) with a biotinylated lectin that recognizes the fucose α 1-2 galactose unit and the possible at least one biotinylated lectin that recognizes the T antigen, in order to obtain colorectal cancer stem cells labeled by the biotinylated lectin that recognizes the fucose α 1-2 galactose unit and the the possible at least one biotinylated lectin that recognizes the T antigen in the histological section,
(e6) placing the histological section obtained in step (d6) in contact with streptavidin, avidin or an anti-biotin antibody bound to a revelation means A,
(f6) revealing the lectin that recognizes the fucose α 1-2 galactose unit and the the possible at least one lectin that recognizes the T antigen,
(g6) detecting and/or quantifying the colorectal cancer stem cells in the histological section obtained at step (f6),
(h6) deducing the recurrence risk and/or the aggressiveness and/or the prognostic value of treating colorectal cancer on the basis of the presence and/or number of colorectal cancer stem cells.

In a preferred embodiment, the present invention concerns an in vitro diagnostic method of the recurrence risk and/or aggressiveness and/or the prognostic values of a colorectal cancer treatment comprising the steps of:
(a7) labeling the colorectal cancer stem cells with an anti-OCT4 antibody in a histological section of a colorectal tissue, in order to obtain colorectal cancer stem cells labeled by the anti-OCT4 antibody in said histological section,
(b7) placing the histological section obtained at step (a7) in contact with a secondary antibody that recognizes the anti-OCT4 antibody bound to a revelation means C, preferably an enzyme capable of reducing a chromogenic substrate, specifically an alkaline phosphatase,
(c7) revealing the anti-OCT4 antibody, preferably with Fast Red® or a BCIP/NBT mixture,
(d7) labeling colorectal cancer stem cells of the histological section obtained at step (c7) with biotinylated UEA-1 or a mixture of UEA-1:Jacalin:ACA in a molar ratio of 6250:160:10, in order to obtain colorectal cancer stem cells labeled by the biotinylated lectin that recognizes the fucose α 1-2 galactose unit and the the possible at least one biotinylated lectin that recognizes the T antigen in the histological section,
(e7) placing the histological section obtained at step (d7) in contact with streptavidin, avidin or an anti-biotin antibody bound to a revelation means A, preferably an enzyme capable of reducing a chromogenic substrate,
(f7) revealing UEA-1 or UEA-1, Jacalin and ACA, preferably with DAB,
(g7) detecting and/or quantifying colorectal cancer stem cells in the histological section obtained at step (f7),
(h7) deducing the recurrence risk and/or aggressiveness and/or prognostic value of the colorectal cancer treatment on the basis of the presence and/or number of colorectal cancer stem cells.

A fifth subject-matter of the present invention concerns a kit, specifically designed to detect and/or quantify colorectal cancer stem cells, comprising:
a biotinylated lectin that recognizes the fucose α 1-2 galactose unit and the possible at least one biotinylated lectin that recognizes the T antigen,
streptavidin bound to a horseradish peroxidase, advantageously incorporated in a signal amplification system,
an anti-OCT4 antibody,
a secondary antibody that recognizes the anti-OCT4 antibody bound to an alkaline phosphatase capable of reducing a chromogenic substrate, advantageously incorporated in an amplification system.

The kit according to the present invention can also contain, in addition to the parts described above, a chromogenic substrate capable of being reduced by horseradish peroxidase and a chromogenic substrate capable of being reduced by alkaline phosphatase.

The kit according to the present invention can also contain buffer solutions, wash solutions, diluents and an instruction book.

In a preferred embodiment, the present invention relates to a kit, specifically designed to detect and/or quantify colorectal cancer stem cells, comprising:
biotinylated UEA-1 and possibly biotinylated ACA and biotinylated Jacalin,
an anti-OCT4 antibody,
a secondary antibody that recognizes the anti-OCT4 antibody, bound to an alkaline phosphatase,
streptavidin bound to a horseradish peroxidase,
possibly DAB,
possibly Fast Red® or a BCIP/NBT mixture,
possibly wash solutions,
possibly diluents,
possibly buffer solutions.

The present invention also concerns the use of a kit as defined above for implementing an in vitro method of detecting and/or quantifying colorectal cancer stem cells in a histological section of colorectal tissue.

The present invention also concerns the use of a kit as defined above in order to implement an in vitro diagnostic method of the recurrence risk and/or aggressiveness of a colorectal cancer to define a prognostic value in order to adapt the treatment of a colorectal cancer.

In a seventh subject-matter, the present invention relates to a method for detecting and/or quantifying in vitro colorectal cancer stem cells in a colorectal biological sample, comprising a step of revealing a lectin that recognizes the fucose α 1-2 galactose unit modified with a revelation means in a colorectal biological sample labeled by said lectin.

The present invention more particularly concerns a method for detecting and/or quantifying in vitro as defined above, comprising the steps of:
(a) labeling the colorectal cancer stem cells with a lectin that recognizes the fucose α 1-2 galactose unit modified with a revelation means in said biological sample, in order to obtain labeled colorectal cancer stem cells in said biological sample,
(b) revealing said lectin in the colorectal biological sample,
(c) detecting and/or quantifying colorectal cancer stem cells labeled by said lectin in said biological tissue.

The present invention thus advantageously relates to a method for detecting and/or quantifying colorectal cancer stem cells in a histological section of colorectal tissue as defined above, in which the revelation means is biotin, comprising the steps of:
(a2) labeling the colorectal cancer stem cells of the histological section with biotinylated lectin that recognizes the fucose α 1-2 galactose unit, in order to obtain colorectal cancer stem cells in said histological section.
(b2) placing the histological section obtained at step (a2) in contact with a revelation means A bound to streptavidin, avidin or an anti-biotin antibody,
(c2) revealing the lectin that recognizes the fucose α 1-2 galactose unit,
(d2) detecting and/or quantifying colorectal cancer stem cells labeled by said lectin in the histological section.

Advantageously, the method described above can be implemented at step (a) with a lectin that recognizes the fucose α 1-2 galactose unit modified with a revelation means and at least one lectin that recognizes the T antigen modified with a revelation means advantageously chosen from ACA, ABA and Jacalin, more advantageously with a mixture of lectins that recognize the T antigen modified with a revelation means chosen from a mixture of ABA and ACA, a mixture of ABA and Jacalin and a mixture of ACA and Jacalin, advantageously ACA and Jacalin.

Advantageously, the lectin mixture consists of a lectin that recognizes the fucose α 1-2 galactose unit:Jacalin modified with a revelation means:ABA modified with a revelation means or ACA modified with a revelation means, in a molar ratio from 625 to 12500:16 to 320:1 to 20, advantageously from 5000 to 7000:50 to 250:5 to 15, specifically 6250:160:10.

Particularly advantageously, step (a) is implemented with a UEA-1 mixture modified with a revelation means, ACA modified with a revelation means and Jacalin modified with a revelation means, preferably in a UEA-1:Jacalin:ACA ratio of 6250:160:10.

The method described above can be preceded by a step of labeling with a second labeling means of cancer stem cells as defined above. In particular, the method described above can be preceded by a step of labeling by an anti-OCT4 antibody.

The present invention thus concerns a method comprising prior to step (a) or (a2) of labeling by the lectin that recognizes the fucose α 1-2 galactose unit the steps of:
(a′1) labeling the colorectal cancer stem cells of said colorectal biological sample with a second labeling means, preferably an anti-OCT4 antibody, in order to obtain colorectal cancer stem cells labeled by an anti-OCT4 antibody in said biological sample,
(a′2) revealing said second labeling means, preferably the anti-OCT4 antibody.

The present invention thus also relates to a method as described above, comprising the steps of:
(a′3) labeling the colorectal cancer stem cells with a second means of labeling colorectal cancer stem cells, preferably an anti-OCT4 antibody, in a histological section of a colorectal tissue, in order to obtain cancer stem cells labeled by said second labeling means in said histological section,
(b3) placing the histological section obtained at step (a3) in contact with a secondary antibody that recognizes the second labeling means, preferably the anti-OCT4 antibody, bound to a revelation means C,
(c3) revealing the second labeling means,
(d3) labeling the colorectal cancer stem cells of the histological section obtained at step (c3) with a biotinylated lectin that recognizes the fucose α 1-2 galactose unit, and the possible at least one biotinylated lectin that recognizes the T antigen, in order to obtain cancer stem cells labeled by the lectin that recognize the fucose α 1-2 galactose unit and possibly labeled by the at least one lectin that recognizes the T antigen in said histological section,
(e3) placing the histological section obtained at step (d3) in contact with a revelation means A bound to streptavidin, avidin or an anti-biotin antibody,
(f3) revealing the lectin that recognizes the fucose α 1-2 galactose unit and the the possible at least one lectin that recognizes the T antigen,
(g3) detecting and/or quantifying the colorectal cancer stem cells labeled by the lectin that recognizes the fucose α 1-2 galactose unit and the second labeling means, preferably the anti-OCT4 antibody in the histological section.

In a particular embodiment, the present invention relates to a method for detecting and/or quantifying colorectal cancer stem cells in a colorectal histological section, comprising the steps of:
(a4) labeling the colorectal cancer stem cells with an anti-OCT4 antibody in a histological section of a colorectal tissue, in order to obtain colorectal stem cells labeled by an anti-OCT4 antibody in said histological section,
(c4) revealing the anti-OCT4 antibody with a chromogenic substrate,
(d4) labeling the colorectal cancer stem cells of the histological section obtained at step (c4) with a biotinylated lectin that recognizes the fucose α 1-2 galactose unit, and the possible at least one biotinylated lectin that recognizes the T antigen, in order to obtain colorectal cancer stem cells labeled by the lectin that recognizes the fucose α 1-2 galactose unit and the possible at least one lectin that recognizes the T antigen in said histological section,
(e4) placing the histological section obtained at step (d4) in contact with streptavidin, avidin or an anti-biotin antibody bound to an enzyme capable of reducing a chromogenic substrate,
(f4) revealing the lectin that recognizes the fucose α 1-2 galactose unit and the the possible at least one lectin that recognizes the T antigen with a chromogenic substrate,
(g4) detecting and/or quantifying colorectal cancer stem cells labeled by the lectin that recognizes the fucose α 1-2 galactose unit, possibly the at least one biotinylated lectin that recognizes the T antigen, and the anti-OCT4 antibody in the histological section.

In a preferred embodiment, the present invention concerns a method for detecting and/or quantifying colorectal cancer stem cells in a colorectal histological section comprising the steps of:
(a5) labeling the colorectal cancer stem cells with an anti-OCT4 antibody in a histological section of a colorectal tissue, in order to obtain colorectal stem cells labeled by an anti-OCT4 antibody in said histological section,
(b5) placing the histological section obtained at step (a5) in contact with a secondary antibody that recognizes the anti-OCT4 antibody bound to an alkaline phosphatase,
(c5) revealing the anti-OCT4 antibody with Fast RED® or a BCIP/NBT mixture,
(d5) labeling the colorectal cancer stem cells of the histological section obtained at step (c5) with biotinylated UEA-1, and possibly biotinylated ACA and biotinylated Jacalin in a UEA-1:Jacalin:ACA ratio of 6250:160:10, in order to obtain colorectal cancer stem cells labeled by UEA-1 and possibly ACA and Jacalin,
(e5) placing the histological section obtained at step (d5) in contact with streptavidin bound to horseradish peroxidase,
(f5) revealing UEA-1 and possibly ACA and Jacalin with DAB,
(g5) detecting and/or quantifying colorectal cancer stem cells labeled by UEA-1 and the anti-OCT4 antibody and possibly ACA and Jacalin in the histological section.

In another embodiment, the general methodology described in the present application can be implemented by using flux cytometry for detecting and/or quantifying colorectal cancer stem cells. In one embodiment, the cancer stem cells can be detected and/or quantified by flux cytometry. This method also allows colorectal cancer stem cells to be isolated by using appropriate equipment.

The biological sample can be a biopsy of a colorectal tumor tissue in which the cells have been dissociated from one another beforehand, for example by means of the product Liberase™, sold by the Roche Diagnostic company, which enables the dissociation of the tissue without affecting the surface glycans.

In order to detect and/or quantify the cells by flow cytometry, the lectin that recognizes the fucose α 1-2 galactose unit is directly or indirectly bound to a fluorophore E. The second labeling means, preferably an anti-OCT4 antibody, is directly or indirectly bound to a fluorophore F, for example to a secondary antibody that recognizes the anti-OCT4 antibody. Advantageously, the fluorophores E and F differ from one another. Preferably, the fluorophore E is Alexa 488 bound to the streptavidin and the lectin that recognizes the fucose α 1-2 galactose unit is biotinylated and the fluorophore F is Alexa 633 directly bound to the anti-OCT4 antibody.

The present invention therefore also relates to a method for detecting and/or quantifying colorectal cancer stem cells as described above, in a colorectal biological sample, in which the detection step is implemented by flow cytometry, with fluorophores as the revelation means.

In one embodiment, the labeling is carried out with a lectin that recognizes the fucose α 1-2 galactose unit modified with a revelation means, advantageously UEA-1, and anti-OCT4 antibody and at least one lectin that recognizes the T antigen modified with a revelation means, advantageously chosen from ACA, ABA and Jacalin or a mixture of lectins that recognize the T antigen modified with a revelation means chosen from an ABA and ACA mixture, an ABA and Jacalin mixture and an ACA and Jacalin mixture, advantageously ACA and Jacalin. Advantageously, the UEA-1:Jacalin:ACA ratio is 6250:160:10.

The present invention relates more particularly to an in vitro diagnostic method of the recurrent risk and/or the aggressiveness and/or the prognostic value of a colorectal cancer treatment comprising the steps of:
(a) labeling the colorectal cancer stem cells in a colorectal biological sample with a lectin that recognizes the fucose α 1-2 galactose unit modified with a revelation means, in order to obtain colorectal cancer stem cells labeled by the lectin that recognizes the fucose α 1-2 galactose unit in said biological sample,
(b) revealing the colorectal cancer stem cells in the colorectal tissue,
(c) detecting and/or quantifying the colorectal cancer stem cells labeled by the lectin that recognizes the fucose α 1-2 galactose unit,
(e) deducing the recurrence risk and/or aggressiveness and/or prognostic value of the colorectal cancer treatment on the basis of the quantity of colorectal cancer stem cells.

Advantageously, the present invention is implemented with a lectin that recognizes the fucose α 1-2 galactose unit with a revelation means and a second means of labeling colorectal cancer cells, preferably an anti-OCT4 antibody.

The inventors in fact revealed that a double labeling led to optimum sensitivity and specificity thus drastically reducing the risk of false positives that could result from labeling cancer non-stem cells or non-cancer stem cells by the lectin that recognizes the fucose α 1-2 galactose unit.

The present invention thus also relates to an in vitro diagnostic method of the recurrence risk and/or aggressiveness in order to define a prognostic value for adapting the treatment of a colorectal cancer comprising the steps of:
(a6) labeling the colorectal cancer stem cells with a second labeling means, preferably an anti-OCT4 antibody in a histological section of a colorectal tissue, in order to obtain labeled colorectal cancer cells in said histological section,
(b6) placing the histological section obtained at step (a6) in contact with a secondary antibody that recognizes the second labeling means bound to a revelation means C,
(c6) revealing the second labeling means, preferably the anti-OCT4 antibody,
(d6) labeling the colorectal cancer stem cells of the histological section obtained at step (c6) with a biotinylated lectin that recognizes the fucose α 1-2 galactose unit and the possible at least one biotinylated lectin that recognizes the T antigen, in order to obtain colorectal cancer stem cells labeled by the biotinylated lectin that recognizes the fucose α 1-2 galactose unit and the the possible at least one biotinylated lectin that recognizes the T antigen in the histological section,
(e6) placing the histological section obtained at step (d6) in contact with streptavidin, avidin or an anti-biotin antibody bound to a revelation means A,
(f6) revealing the lectin that recognizes the fucose α 1-2 galactose unit and the the possible at least one lectin that recognizes the T antigen,
(g6) detecting and/or quantifying the colorectal cancer stem cells in the histological section obtained at step (f6),
(h6) deducing the recurrence risk and/or the aggressiveness and/or the prognostic value of treating colorectal cancer on the basis of the presence and/or number of colorectal cancer stem cells.

In a preferred embodiment, the present invention concerns an in vitro diagnostic method of the recurrent risk and/or aggressiveness and/or the prognostic value of a colorectal cancer treatment comprising the steps of:
(a7) labeling the colorectal cancer stem cells with an anti-OCT4 antibody in a histological section of a colorectal tissue, in order to obtain colorectal cancer stem cells labeled by the anti-OCT4 antibody in said histological section,
(b7) placing the histological section obtained at step (a7) in contact with a secondary antibody that recognizes the anti-OCT4 antibody bound to a revelation means C, preferably an enzyme capable of reducing a chromogenic substrate, specifically an alkaline phosphatase,
(c7) revealing the anti-OCT4 antibody, preferably with Fast Red® or a BCIP/NBT mixture,
(d7) labeling the colorectal cancer stem cells of the histological section obtained at step (c7) with biotinylated UEA-1 or a mixture of UEA-1:Jacalin:ACA in a molar ratio of 6250:160:10, in order to obtain colorectal cancer stem cells labeled by the biotinylated lectin that recognizes the fucose α 1-2 galactose unit and the possible at least one biotinylated lectin that recognizes the T antigen in the histological section,
(e7) placing the histological section obtained at step (d7) in contact with streptavidin, avidin or an anti-biotin antibody bound to a revelation means A, preferably an enzyme capable of reducing a chromogenic substrate,
(f7) revealing UEA-1 or UEA-1, Jacalin and ACA, preferably with DAB,
(g7) detecting and/or quantifying the colorectal cancer stem cells in the histological section obtained at step (f7),
(h7) deducing the recurrence risk and/or aggressiveness and/or prognostic value of the colorectal cancer treatment on the basis of the presence and/or number of colorectal cancer stem cells.

### DESCRIPTION OF THE FIGURES

FIG. 1 represents histological sections in series of a healthy tissue labeled by UEA1 (top Figure), an anti-OCT4 antibody (middle Figure) and doubly labeled by UEA-1/anti-OCT4 antibody (bottom Figure). In the healthy tissue, the absence of color due to double labeling can be seen.

FIG. 2 represents histological sections in series of a tumor tissue labeled by UEA1 (top Figure), an anti-OCT4 antibody (middle Figure) and doubly labeled by UEA-1/anti-OCT4 antibody (bottom Figure). In this tumor tissue, a significant dark staining can be seen (brown in the image obtained in practice) resulting from a double labeling by UEA-1 and the anti-OCT4 antibody.

FIG. 3 shows the image of a healthy tissue double labeled by UEA-1/streptavidin conjugated with Alexa Fluor 488 and an anti-OCT3 antibody conjugated with Alexa Fluor 594. The absence of fluorescence indicates the absence of colorectal cancer stem cells.

FIG. 4 shows the image of a tumor tissue doubly labeled by UEA-1/streptavidin conjugated with Alexa Fluor 488 and an anti-OCT4 antibody conjugated with Alexa Fluor 594. The light areas on the image are due to the double labeling of cancer stem cells by UEA-1/streptavidin conjugated with Alexa Fluor 488 and the anti-OCT4 antibody with Alexa Fluor 594. The double labeling reveals the presence of a large number of colorectal cancer stem cells.

FIG. 5 shows the adjusted confocal microscopy image after using the z-stack method obtained by double labeling of a healthy tissue with UEA-1/streptavidin conjugated with Alexa Fluor 488 and an anti-OCT4 antibody conjugated with Alexa Fluor 594. The top left-hand quadrant corresponds to the cells labeled by the anti-OCT4 antibody, the bottom left-hand quadrant corresponds to the unlabeled cells and the bottom right-hand quadrant corresponds to the cells labeled by UEA-1. The top right-hand quadrant corresponds to the cells doubly labeled by UEA-1 and the anti-OCT4 antibody. The absence of doubly labeled cells confirms that the tissue contains no colorectal stem cells.

FIG. 6 shows the adjusted confocal microscopy image after using the z-stack method obtained by double labeling of a tumor tissue with UEA-1/streptavidin conjugated with Alexa Fluor 488 and an anti-OCT4 antibody conjugated with Alexa Fluor 594. The top left-hand quadrant corresponds to the cells labeled by the anti-OCT4 antibody, the bottom left-hand quadrant corresponds to the unlabeled cells and the bottom right-hand quadrant corresponds to the cells labeled by UEA-1. The top right-hand quadrant corresponds to the cells doubly labeled by UEA-1 and the anti-OCT4 antibody. The presence of cells in the top right-hand quadrant indicates the presence of 16.6% colorectal cancer stem cells.

FIG. 7 shows the image of a healthy tissue doubly labeled by the anti-OCT4 antibody and UEA-1 (top image) and the image of a tumor tissue double labeled by the anti-OCT4 antibody and UEA-1 in which a large number of colorectal cancer stem cells is present (bottom image). The double labeling has no healthy tissue whereas numerous cells are labeled in the tumor tissue (dark brown patches in the image obtained in practice).

FIGS. 8 to 11 show the results of histochemical labeling on sections of tumor tissues of patients suffering from colorectal cancers at different stages of development, corresponding to the retrospective clinical study described in Example 3.

FIG. 8 shows the results obtained for a patient suffering from a stage I colorectal cancer having survived (favorable case, patient I) and a patient suffering from a stage I colorectal cancer not having survived (unfavorable case, patient II). The labeling was carried out either with the anti-OCT4 antibody (Ia and IIa), or with the anti-OCT4 antibody and a mixture of UEA-1/ACA/Jacalin lectins (Ib and IIb), or with a mixture of UEA-1/ACA/Jacalin lectins (Ic and IIc).

FIG. 9 shows the results obtained for a patient suffering from a stage II colorectal cancer having survived (favorable case, patient I) and a patient suffering from a stage II colorectal cancer not having survived (unfavorable case, patient II). The labeling is carried out either with the anti-OCT4 antibody (Ia and IIa), or with the anti-OCT24 antibody and UEA-1 (Ib and IIb) or with UEA-1 (Ic and IIc).

FIG. 10 shows the results obtained for a patient suffering from a stage III colorectal cancer having survived (favorable case, patient I) and of a patient suffering from a stage III colorectal cancer not having survived (unfavorable case, patient II). The labeling is achieved either with the anti-OCT4 antibody (Ia and IIa), or with the anti-OCT4 antibody and a mixture of UEA-1/ACA/Jacalin lectins (Ib and IIb) or with a mixture of UEA-1/ACA/Jacalin lectins (Ic and IIc).

FIG. 11 shows the results obtained for a patient suffering from a stage IV colorectal cancer having survived with a chemotherapy treatment (favorable case, patient I) and of a patient suffering from a stage IV colorectal cancer not having survived (unfavorable case, patient II). The labeling is carried out either with the anti-OCT4 antibody (Ia and IIa), or with the anti-OCT24 antibody and UEA01 (Ib and IIb) or with UEA-1 (Ic and IIc).

### EXAMPLES

### Example 1

### Double Labeling Visible on a Histological Section

Double Labeling Protocol with Lectin(s)/OCT4 in Paraffin:

Equipment used: Blocks of paraffin, Ice, Microtome, Superfrost® Slides, Automated Bond Max Stainer (Leica Microsystems) with computer, Leica Consumables (alcohol, wash buffer, ER1 buffer, dewax buffer, labels, coverslips, tubes), 5% PBS-BSA buffer, Anti-OCT4 antibody (ThermoFisher Scientific), Bond Polymer Refine Red Detection Kit (Leica), Leica mounting medium, slides and microscope.

The paraffin blocks containing the CRC samples from each of the patients identified by their number (given by Pathological Anatomy Department) were packed in ice for about 1 hour to cool them so as to facilitate their microtome sectioning to a thickness of 4 μm.

So-called “superfrost” slides, used to achieve maximum adhesion of the sectioned tissue, were identified by the same numbers as those present on the blocks. A drop of water was placed in the center of each of these slides.

Microtome sections were made and placed on the previously deposited drop of water. The slides were then placed on a hot plate at 37° C. to facilitate their adhesion and the excess water was removed. All of the slides made were placed in an oven at 37° C. to dry.

The rest of the manipulation was handled by the Leica Automated Bond Max stainer connected to a computer running software to control the stainer.

During the time that the slides were in the oven, the double labeling manipulation preparations were carried out, starting by checking the level in the stainer of each of the products needed to perform the manipulation, then identifying the slides by their same numbers, using the stainer operating software. Labels enabling a standardized protocol were generated. The dilution of the antibodies and their quantity were calculated and the various kits required were prepared. Note that each of the products used had to be scanned and the level reset to zero before each experiment performed.

The labels were then affixed to their corresponding slides on being taken out of the oven and coverslips were placed onto each one to allow the product to be present over the entire surface of the slide thanks to contact properties.

The slide rack was placed in the stainer and, after each of the elements were recognized by the reader and the slides identified by their barcodes on the labels, manipulation was begun.

Firstly, the paraffin was removed by heating, thanks to the Leica Dewax product, consequently enabling access to the antibodies. This step, as well as all the others, was followed by three washes in previously diluted Bond Wash Solution 10X.

This step was followed by a pretreatment for 5 min with Leica ER1 buffer, which has a pH of 6, to demask the antigens to be obtained during this double labeling procedure.

The anti-OCT4 antibody (rabbit, ThermoFisher Scientific) was first applied at 1:500 for 20 min, dilution being in 5% PBS-BSA to prevent non-specific bindings due to its saturation power.

The Leica Bond Polymer Refine Red Detection kit (anti-rabbit antibody conjugate with alkaline phosphatase polymer adaptor) then enabled this antibody to be revealed thanks to Fast Red® in the red visible spectrum.

The biotinylated lectin or a mixture of biotinylated lectins, i.e. UEA-1 at 1:40 or the 1:3 mixture of lectins composed of 1:40 UEA-1 with 1:400 Jacalin and 1:25000 ACA, always using 5% PBS-BSA diluent, was applied for 20 min. Note that the order of labeling is important to ensure that the manipulation proceeds satisfactorily.

The Bond Intense R Detection kit, thanks to the intervention of a streptavidin-HRP playing the role of secondary antibody enabled, due to its properties, this or these biotinylated lectins to be revealed in brown thanks to the properties of DAB recognizing the biotin/streptavidin-HRP complex.

A bluish counterstaining step, thanks to the presence of hematoxylin, was then performed for 7 min to ensure that the whole sample could be identified.

The slides were removed from the stainer. The sections were then rehydrated by immersing the slides manually in an alcohol bath twice for 5 min. This rehydration step was followed by a toluene bath, also for 5 min.

The slides could then be mounted by applying a drop of mounting medium (Leica). This particular medium is a bioadhesive that prevents the respective labels from deteriorating and dissolving, particularly the red ones.

Lastly, the slides were observed under the microscope and images were taken at a magnification of 20.

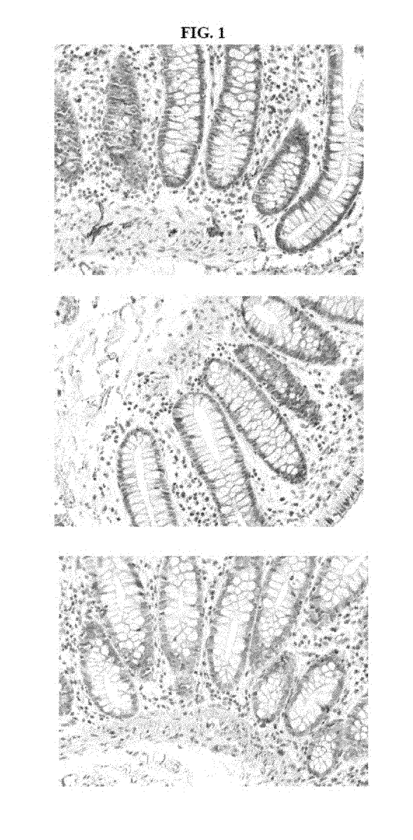

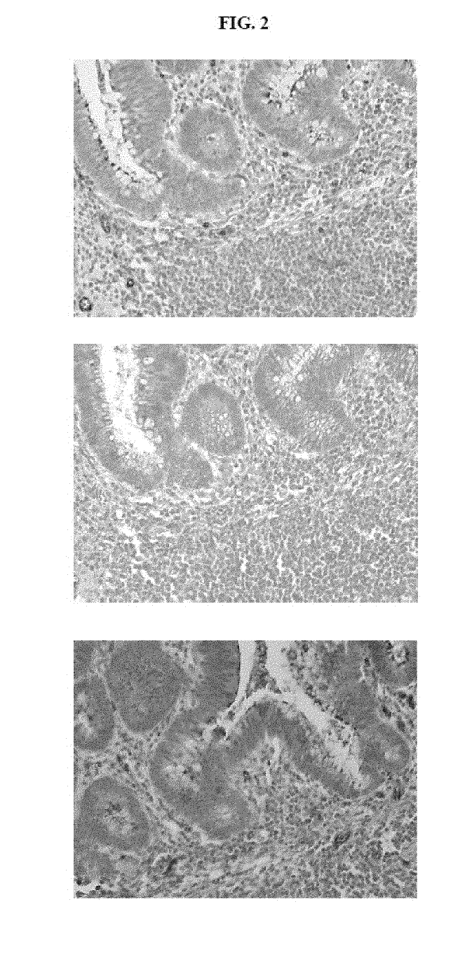

The results are shown in FIGS. 1 and 2.

The double labeling of a healthy tissue by UEA-1 and by an anti-OCT4 antibody showed the absence of double labeling of the cells. The absence of colorectal cancer stem cells in this tissue can therefore be deduced.

Several cell types exist in a tumor tissue: non-stem tumor cells, non-tumor stem cells, tumor stem cells and non-tumor non-stem cells. Labeling a tumor tissue with UEA-1 alone or UEA-1 and an anti-OCT4 antibody proves the ability of the lectin UEA-1 to selectively label colorectal cancer stem cells (darker areas). OCT4 is capable of labeling (tumor and non-tumor) stem cells and confirms that the cells labeled by UEA-1 are colorectal cancer stem cells.

### Example 2

### Double Fluorescent Labeling on a Histological Section

Some 4 to 6 μm sections were incubated:
in the presence of an anti-OCT4 antibody (rabbit, 1:200 dilution) overnight at 4° C., then the sections were washed three times with PBS and incubated for 1 hour with an anti-rabbit secondary antibody conjugated with Alexa Fluor 594 (ThermoFisher Scientific);
in the presence of biotinylated UEA-1 (1:200 dilution) for 20 to 30 min at ambient temperature, then the sections were washed three times with PBS and revealed by streptavidin 488 conjugated with Alexa Fluor 488 for 30 min;
in the presence of anti-OCT4 antibody (rabbit, 1:200 dilution) overnight at 4° C., then the sections were washed three times with PBS and incubated for 1 hour with an anti-rabbit secondary antibody conjugated with Alexa Fluor 594 (ThermoFisher Scientific). The sections were then incubated in the presence of biotinylated UEA-1 (1:200 dilution) for 20 to 30 min at ambient temperature, washed three times with PBS and revealed by streptavidin conjugated with Alexa Fluor 488 for 30 min;

After three washes in PBS, the nuclei were stained with DAPI (1:10000) for 5 minutes. After 3 additional washes with DAPI in water, the sections were mounted on slides and examined under an LSM 510 META confocal microscope (Zeiss) using the associated software.

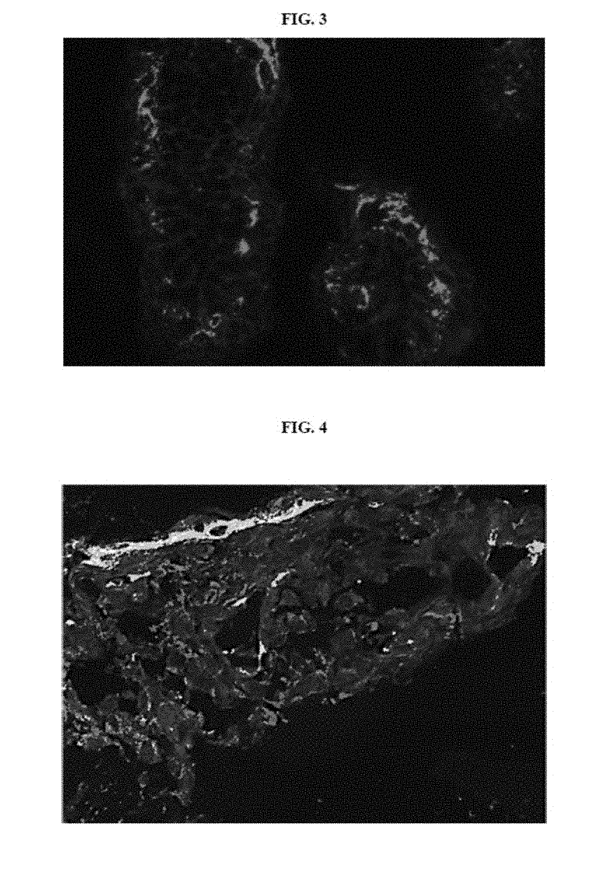

The results are shown in FIGS. 3 and 4.

In a healthy tissue (FIG. 3), a double labeling was not observed. In a tumor tissue, by contrast, a double labeling of the colorectal cancer stem cells can be shown (FIG. 4, light area of the image).

Cells incubated with an anti-OCT4 antibody conjugated with Alexa Fluor 594 and biotinylated UEA-1/streptavidin conjugated with Alexa Fluor 488 were analyzed by confocal microscopy and the image adjusted by the z-stack method.

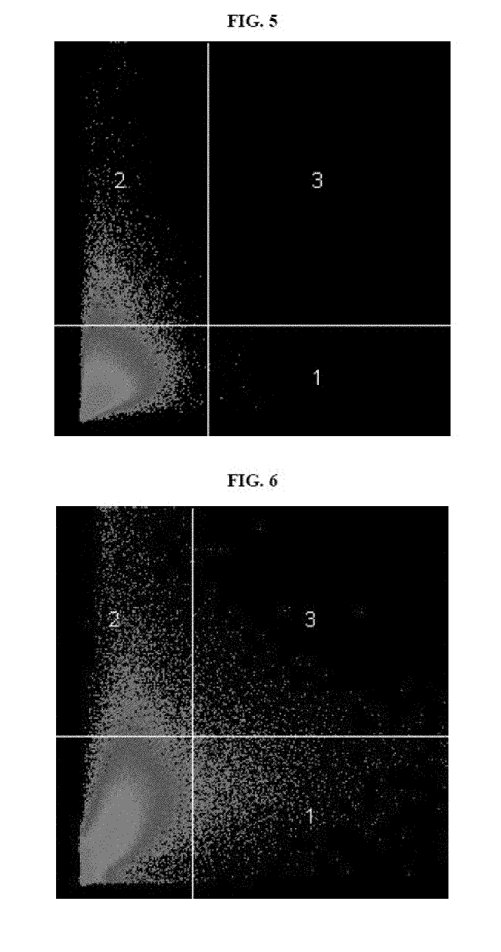

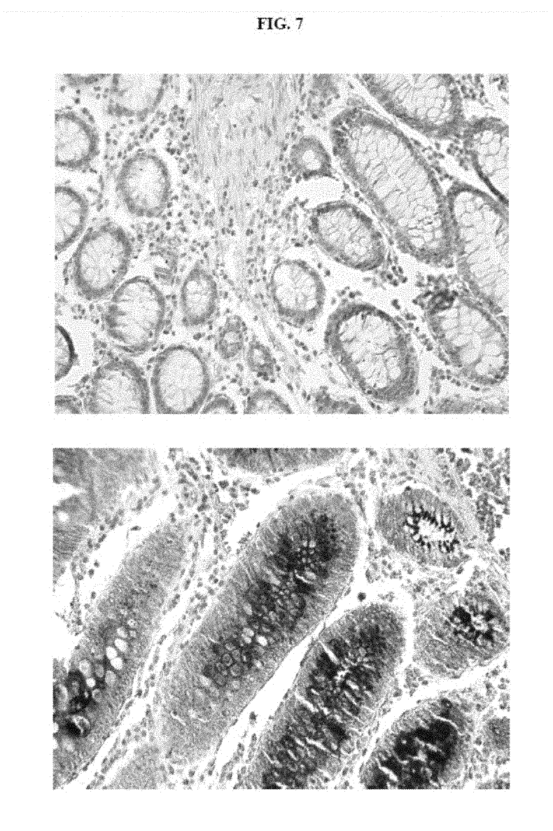

The results are shown in FIGS. 5 and 6.

In the healthy tissue (FIG. 5), no cell is doubly labeled, indicating the absence of colorectal cancer stem cells.

In the tumor tissue, analysis of the adjusted image enables cells that are doubly labeled by UEA-1 and the anti-OCT4 antibody to be detected. The quantity of these cells represents 16.6% of the total number of cells.

On the basis of these data, it is possible to conclude that the tumor contains colorectal cancer stem cells capable of leading to a recurrence. The aggressiveness of the tumor being proven, as well as the recurrence risk, it can therefore be concluded that the treatment must be followed and duly adapted.

### Example 3

A retrospective clinical study on the tumor tissues of 100 patients suffering from colorectal cancers at different stages of development was carried out.

The object of this study was to validate the colorectal cancer aggressiveness test by an immunohistochemical measurement using specific lectins of the cancer stem cells. The samples were deliberately chosen from a cohort of patients dating back 5 years in order to know the survival rates of the patients in the study over 60 months. Monitoring patient outcomes over 5 years also makes it possible to go beyond the action time of chemotherapy treatments which is estimated to be 3 years.

For each stage of cancer two examples are presented. The favorable case of a negative sample for the cancer stem cell labels and for which the patient survived after 60 months. The unfavorable case of a positive sample for the cancer stem cell labels and for which the patient did not survive.

For the examples of stage I colorectal cancer, the patients had the tumor removed and did not receive chemotherapy. This is a conventional protocol for the treatment of stage I colorectal cancer.

For the examples of stage II colorectal cancer, the patients had the tumor removed and did not receive chemotherapy. This is a conventional protocol for the treatment of stage II colorectal cancer presenting a sufficient number of peripheral ganglia.

For the examples of stage III colorectal cancer, the patients had the tumor removed and did not receive chemotherapy. These are specific cases of stage III colorectal cancer treatment and usually patients also undergo chemotherapy in addition to surgery.

For the examples of stage IV colorectal cancer, the patients had the tumor removed and systematically received chemotherapy treatment. In the unfavorable example shown here, the patient died before receiving chemotherapy treatment.

Labeling was performed as follows:

Stage I Colorectal Cancer

Double labeling: OCT4+ Mixture of lectins (UEA-1, Jacalin, ACA)

Glycosylated Label: Mixture of lectins (UEA-1, Jacalin, ACA)

Stage II Colorectal Cancer

Double labeling: OCT4+ UEA-1 lectin only

Glycosylated Label: UEA-1 lectin only

Stage III Colorectal Cancer

Double labeling: OCT4+ Mixture of lectins (UEA-1, Jacalin, ACA)

Glycosylated Label: Mixture of lectins (UEA-1, Jacalin, ACA)

Stage IV Colorectal Cancer

Double labeling: OCT4+ UEA-1 lectin only

Glycosylated Label: UEA-1 lectin only

For each stage (I, II and III) the analysis enabled groups of patients to be identified that had positive and negative test results (presenting or not presenting cancer stem cells). The test provided additional information for the practitioner and envisaged a specific treatment or monitoring for patients with positive results.

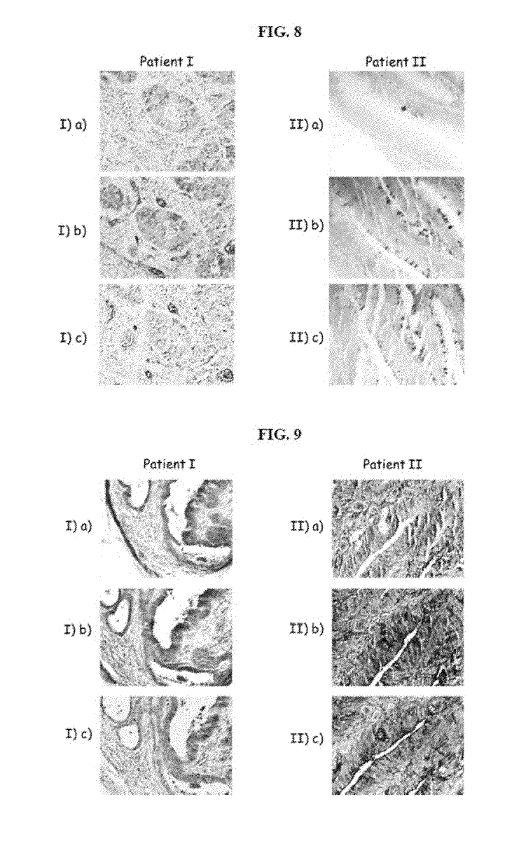

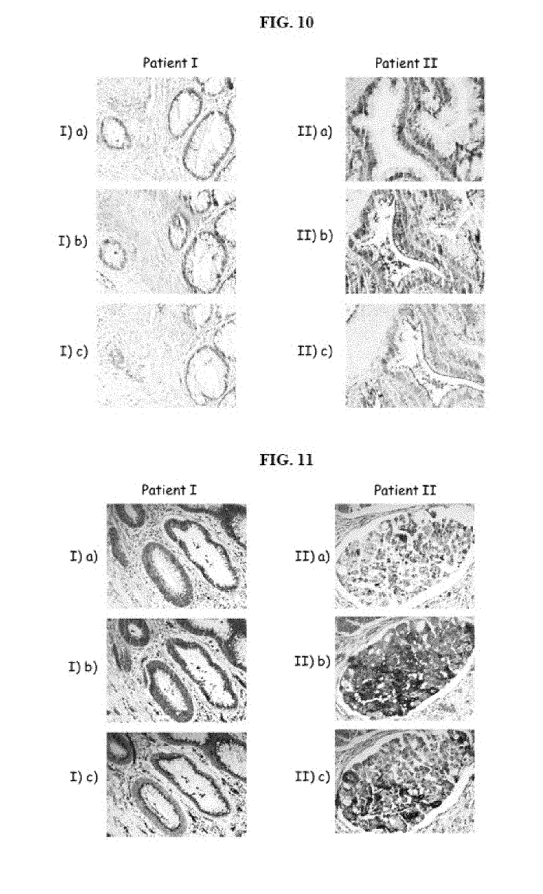

The results are shown in FIGS. 8 to 11.

1-12. (canceled)

13. A method of detecting or quantifying colorectal cancer stem cells, the method comprising a step of contacting a sample comprising colorectal cancer stem cells with a lectin that binds fucose α 1-2 galactose.

14. The method of claim 13, wherein the lectin that binds fucose α 1-2 galactose is selected from the group consisting of Ulex Europaeus Agglutinin 1 (UEA-1) and Trichosanthes Japonica Agglutinin II (TJA-II).

15. The method of claim 13, comprising an additional step of contacting the sample comprising colorectal cancer stem cells with at least one labelling agent that binds colorectal cancer stem cells

16. The method of claim 15, wherein the at least one labelling agent that binds colorectal cancer stem cells comprises an anti-OCT4 antibody

17. The method of claim 13, further comprising the stepa of contacting the sample comprising colorectal cancer stem cells with an anti-OCT4 antibody and contacting the sample comprising colorectal cancer stem cells with a lectin that binds to the T antigen.

18. The method of claim 17, wherein the lectin that binds to the T antigen is selected from the group consisting of Amaranthus Caudatus Lectin (ACA), Agaricus Bisporus Agglutinin (ABA), and Jacalin.

19. The method of claim 13, wherein a detectable label A of the lectin that binds fucose α 1-2 galactose is conjugated to streptavidin and the lectin that binds fucose α 1-2 galactose is biotinylated.

20. The method of claim 19, wherein the detectable label A conjugated to streptavidin is selected from the group consisting of a chromophore, a fluorophore, and an enzyme capable of reducing a chromogenic substrate B

21. The method of claim 16, wherein a detectable label C of the anti-OCT4 antibody is bound to a secondary antibody recognizing the anti-OCT4 antibody.

22. The method of claim 21, wherein the detectable label C is selected from the group consisting of a chromophore, a fluorophore, an enzyme capable of reducing a chromogenic substrate D.

23. The method of claim 13, wherein the sample comprising colorectal cancer stem cells is a histological section.

24. The method of claim 23, wherein the sample comprising colorectal cancer stem cells is contacted with:
(a) biotinylated UEA-1;
(b) horseradish peroxidase conjugated with streptavidin;
(c) diaminobenzidine;
(d) a secondary antibody recognizing the anti-OCT4 antibody conjugated with alkaline phosphatase; and
(e) 3-amino-4-methoxybenzamide.

25. The method of claim 13, further comprising the steps of revealing said lectin in the colorectal biological sample and detecting or quantifying colorectal cancer stem cells labeled with said lectin in said biological tissue.

26. A method for selecting a therapeutic approach for treating colorectal cancer in a subject, comprising the steps of:
(a3) staining colorectal cancer stem cells with an anti-OCT4 antibody in a histological section of colorectal tissue to obtain colorectal cancer stem cells in said histological section;
(b3) contacting the histological section obtained in step (a3) with a secondary antibody recognizing the anti-OCT4 antibody linked to a detectable label C;
(c3) revealing of the anti-OCT4 antibody;
(d3) staining of the colorectal cancer stem cells of the histological section obtained in step (c3) with a biotinylated lectin that binds fucose α 1-2 galactose to obtain colorectal cancer stem cells labeled with biotinylated lectin that binds α 1-2 galactose fucose;
(e3) contacting the histological section obtained in step (d3) with streptavidin, avidin or an anti-biotin antibody linked to a detectable label A;
(f3) revealing of the lectin that binds fucose α 1-2 galactose;
(g3) detecting or quantifying the colorectal cancer stem cells in the histological section obtained in step (f3); and
(h3) deducing the risk of recurrence or aggressiveness of colorectal cancer to define a prognostic value for the therapeutic approach for treating colorectal cancer from the presence or the number of colorectal cancer stem cells in the histological sample.

27. The method of claim 26, wherein step (d3) further comprises staining the colorectal cancer stem cells of the histological section with at least one biotinylated lectin recognizing the T antigen to obtain colorectal cancer stem cells labeled with the at least one biotinylated lectin recognizing the T antigen in the histological section.

28. The method of claim 27, wherein step (f3) further comprises revealing the at least one lectin recognizing the T antigen.

29. A kit comprising: a biotinylated lectin that recognizes the fucose α 1-2 galactose unit, streptavidin bound to a horseradish peroxidase, an anti-OCT4 antibody, and a secondary antibody that recognizes the anti-OCT4 antibody bound to alkaline phosphatase.

30. The kit of claim 29, further comprising at least one biotinylated lectin that binds to the T antigen.

31. The kit of claim 29, further comprising at least one biotinylated lectin that binds to the T antigen and wherein the lectin that binds to the T antigen is selected from the group consisting of ACA, ABA, and Jacalin.
